(19) United States
(12) Patent Application Publication
(10) Pub. No.: US 2022/0101985 A1
(43) Pub. Date: Mar. 31, 2022

(54) EFFICIENTLY SCHEDULING REFERRAL APPOINTMENTS

(71) Applicant: International Business Machines Corporation, Armonk, NY (US)

(72) Inventors: ITAI GORDON, Modiin (IL); Kristin E. McNeil, Charlotte, NC (US); Shlomit Avrahami, Jerusalem (IL); ROTEM BROSH, Be'er Ya'akov (IL); SHIRAN YOSEF, Petach Tiqwa (IL)

(21) Appl. No.: 17/037,907

(22) Filed: Sep. 30, 2020

(51) Int. Cl. G16H 40/20; G16H 10/60; G06Q 10/10; G16H 50/20; G06F 40/40; G06K 9/00

(52) U.S. Cl. CPC G16H 40/20; G16H 10/60; G06K 9/00463; G16H 50/20; G06F 40/40; G06Q 10/1095

(57) ABSTRACT

Embodiments herein disclose computer-implemented methods, computer program products and computer systems for efficiently scheduling referral appointments. The computer-implemented method may include receiving text data corresponding to a referral request; determining patient data based on the text data comprising patient demographic data, patient complaint data, patient medication data, and patient history data; determining doctor data based on the text data comprising sending doctor data and receiving doctor; determining a patient treatment time based on the patient data and the receiving doctor data; sending an appointment request including the patient treatment time to a receiving doctor computing device; and receiving an appointment confirmation including the patient treatment time, wherein the appointment confirmation indicates acceptance of the appointment request by the receiving doctor. Text fields having respective text field entries may be identified in a medical referral document using an NLP engine for the referral request.

### BACKGROUND

The present invention relates generally to the field of scheduling appointments, and more particularly to efficiently scheduling referral appointments using a Natural Language Processing (NLP) engine.

Today, many specialized physician medical practices are inundated with receiving and treating new and existing patients. This influx of patients is usually a result of referrals from primary care physicians from general medicine practices, either referred from in-network or out-of-network providers. With so many new appointments being made to a particular specialized medical practice, long wait periods are an inevitable obstacle in the way of the patient seeking specialized treatment. In order to maximize their time and to ensure that they are the appropriate medical professional for the referred patient, many specialized physicians require a reference letter or a referral from a referring physician before they agree to treat a patient. Sometimes, the referring physician may call or ask the patient to call the receiving physician to schedule the appointment.

The referral letter may be handwritten, typed using a typewriter, or generated using a computer. The referral letter includes medical details about why the patient should be treated by the specialized physician. Usually, it is difficult to accurately predict how much time a patient may require since each patient has different medical and personal circumstances and each medical physician's practice has its own unique characteristics and methods in which they accept and treat patients. In addition to the patient and physician's unique needs and trends, the anticipated treatment time may vary based on the type of treatment that is anticipated when the referral request is made. Therefore, the receiving physician may determine that the patient may need more or less time depending on the information presented and available to factor into the decision. Nonetheless, the referral process may take unnecessary amounts of time given the busy schedules of both the referring and receiving physicians, not to mention the patient's busy work/life schedule.

### SUMMARY

The present invention is described in various embodiments disclosing methods, computer program products, and computer systems for scheduling a referral appointment. One embodiment of the present disclosure is a computer-implemented method for scheduling a referral appointment, wherein the computer-implemented method may include one or more processors configured for receiving text data corresponding to a referral request; determining patient data based on the text data comprising one or more of patient demographic data, patient complaint data, patient medication data, and patient history data; determining doctor data based on the text data comprising one or more of sending doctor data and receiving doctor data; determining a patient treatment time based on the patient data and the receiving doctor data; sending an appointment request including the patient treatment time to a computing device associated with the receiving doctor data; and receiving an appointment confirmation including the patient treatment time, wherein the appointment confirmation indicates acceptance of the appointment request.

In an embodiment, the computer-implemented method may further include one or more processors configured for identifying text fields having respective text field entries represented in a medical referral document image comprising the referral request; and generating the text data using the text fields and the respective text field entries.

In an embodiment, the patient data may be determined using a Natural Language Processor (NLP) engine to extract the identified text fields corresponding to a patient and identify the respective text field entries corresponding to the patient as one of the patient demographic data, the patient complaint data, the patient medication data, or the patient history data as the patient data.

In another embodiment, the computer-implemented method, wherein determining the doctor data may further include extracting the identified text fields corresponding to a sending doctor and a receiving doctor using a Natural Language Processor (NLP) engine; and identifying the respective text field entries corresponding to the sending doctor and the receiving doctor as the doctor data using the NLP engine.

In an embodiment, the computer-implemented method may further include one or more processors configured for determining doctor schedule data based on the receiving doctor data, wherein the patient treatment time is further determined based on the doctor schedule data. The computer-implemented method may further include one or more processors configured for determining patient schedule data based on the patient demographic data, wherein the patient treatment plan is further determined based on the patient schedule data.

In an embodiment, determining the patient treatment time may include training a machine learning model with data sets including the patient history data and receiving doctor history data; providing the patient data and the receiving doctor data to the trained machine learning model; and predicting the patient treatment time as a quantity of time represented by output data of the machine learning model.

### BRIEF DESCRIPTION OF THE DRAWINGS

FIG. 1 depicts a block diagram of a distributed data processing environment for efficiently scheduling referral appointments, in accordance with an embodiment of the present invention;

FIG. 2 depicts an example embodiment of a medical referral document image to efficiently schedule referral appointments, in accordance with an embodiment of the present invention;

FIG. 3 depicts a block diagram of a system for efficiently scheduling referral appointments using a machine learning model, in accordance with an embodiment of the present invention;

FIG. 4 depicts a flow chart of steps of a computer-implemented method for efficiently scheduling referral appointments, in accordance with an embodiment of the present invention; and

FIG. 5 depicts a block diagram of a computing device of the distributed data processing environment of FIG. 1, in accordance with an embodiment of the present invention.

### DETAILED DESCRIPTION

The present invention addresses the problem of inefficiently scheduling a referral appointment from a referring physician (i.e., sending doctor) to a specialist physician (i.e., receiving doctor). Computer-implemented methods, systems, and computer program products are described that facilitate better appointment scheduling by physicians by using Natural Language Processing (NLP) to analyze reference letters or referral letters for use with patient data and doctor data to predict the best date and time a patient needs for their treatment. For example, an embodiment may include an NLP engine or NLP component configured to receive data of a scanned document and extract text from the document to identify medical concepts that are relevant to an amount of time needed for a particular treatment.

In an embodiment, the one or more processors may be configured to transmit data corresponding to an appointment request to a computing device associated with the receiving doctor. The appointment request may be generated by processing text data corresponding to a referral request, as described above herein. The appointment request may also come in the form of a telephone call from a user device associated with the patient to a computing device associated with the receiving doctor. The text data may include patient data and doctor data, wherein the doctor data may include sending doctor data and receiving doctor data.

In an embodiment, the one or more processors may be configured to determine patient data and doctor data based on the identified text extracted from the document. Patient data may include patient demographics (e.g., name, age, weight, height, address, contact information), patient complaint data (e.g., symptoms, conditions), patient medication data (e.g., prescription medication, over-the-counter medication) and patient history data (e.g., past treatment types, times of treatments, past appointments with a doctor). Doctor data may include sending doctor data (i.e., data about the doctor making the referral) and receiving doctor data (i.e., data about the specialist doctor receiving the referred patient). Both sending doctor data and receiving doctor data may include respective doctor demographics, doctor specialization data, doctor appointment time data and other information relevant to the doctor's practice.

Once the patient data and the doctor data are received, the one or more processors may be configured to determine patient schedule data and doctor schedule data by receiving respective schedule availabilities using the patient data and the doctor data, respectively. The NLP engine described above may be a machine learning model that may be trained with historical patient data and historical doctor data to produce a trained model for predicting the right amount of time to treat the patient for a particular issue. The one or more processors may be further configured for sending the appointment request to the receiving doctor and receiving an appointment confirmation or new proposed time from the receiving doctor. Once the appointment confirmation is received by the one or more processors, the computer-implemented method may send the appointment confirmation to a computing device associated with the patient. The machine learning model may be trained with data corresponding to concepts and properties about the patient that could assist in assessing how much time it would take for the receiving doctor to treat the patient. The machine learning model may also be trained with data corresponding to a particular medical issue or medical condition to predict the amount of time a later treatment would take based on the training data. Patient data and receiving doctor data may be used in conjunction with the predicted amount of time the treatment should take to determine an adjusted time the later treatment would take. The adjustment would be determined based on the specifics of the patient and the doctor's practice that may increase or decrease the time the particular treatment would take without considering the specifics.

In another embodiment, the one or more processors may be configured to determine if the predicted appointment time would be sufficient for the patient's and doctor's needs based on the patient data and the doctor data. The predicted appointment time may be compared to previous appointment times for similar treatment types and identify discrepancies that may exist between the results. For example, the patient may have needed additional time in a previous visit that has similar circumstances as the upcoming appointment, wherein the doctor or patient may provide an additional input configured to manually update the predicted appointment time and correct the prediction accordingly.

In an embodiment, the NLP component may be a rules-based model configured to process unstructured text data to identify medical terminology and related concepts. The rules-based NLP component may be in the form of an Unstructured Information Management Architecture (UIMA), which may be configured to analyze large volumes of unstructured information in order to learn information that is relevant to the study. For example, the NLP component may be an UIMA application configured to analyze large volumes of unstructured information to discover and learn knowledge relevant to the study. The UIMA application may ingest plain text and identify entities, such as persons, places, organizations; or relations, such as works-for or located-at.

In another embodiment, the one or more processors may be configured to schedule the appointment based on the doctor schedule data and the patient schedule data, wherein an available time is identified that accommodates the predicted appointment time determined as described above herein. Doctor schedule data may include rules established by the receiving doctor to establish baselines for when an appointment for a particular type of treatment should be scheduled. The rules may also define a minimum or maximum amount of time to allocate for a patient based on the patient data or based on the treatment type, or both. The rules may also define particular times of the day in which an appointment should or should not be scheduled based on the patient data or based on the treatment type, or both. Similarly, patient schedule data may include rules established by the patient to establish baselines for when an appointment for a particular type of treatment should be scheduled. The patient schedule data may also include rules that define the maximum amount of time a patient prefers to be seen by a doctor or days in which no appointment can be requested or should only be requested.

In another embodiment, the one or more processors may be configured to measure or record actual treatment times using one or more sensors configured to detect the time when the receiving doctor enters the treatment room or examination room. One or more sensors may also be configured to determine when the receiving doctor accesses the patient records, indicating the beginning or some portion of the appointment. A notification may be sent to a user device associated with the patient to notify the patient of the receiving doctor's activity. Such notifications can provide real-time notifications to the patient to lower uncertainty surrounding doctor visits.

In another embodiment, the one or more processors may be configured to receive updates from the receiving doctor, wherein the updates may update the data corresponding to the appointment. For example, if the appointment was scheduled to begin at 9:00 AM, but the receiving physician did not begin the appointment until 9:15 AM, then the one or more processors may be configured to receive input from a computing device associated with the receiving doctor corresponding to an appointment begin time of 9:15 AM, thereby updating the actual time the appointment began. As another example, if the appointment was scheduled to have a duration time of 1.0 hours and the appointment actually lasted 1.5 hours, then the one or more processors may be configured to receive input from a computing device associated with the receiving doctor corresponding to a duration time of 1.5 hours, thereby updating the actual time the appointment lasted. Any modification to variable data may be updated by the receiving doctor or the patient or either of their agents.

In another embodiment, if no available appointment time is determined based on the patient schedule data, the doctor schedule data, and other rules set by the patient and the receiving doctor, then the one or more processors may be configured to identify a list of appointments and sort the list of appointments based on criteria set by the receiving doctor to minimize the amount of time a treatment should take on a patient-to-patient basis. For example, if a set criterion identifies a number of patients who prefer appointments during a certain time of the day (e.g., morning, afternoon, late afternoon), and sorts the patients according to that criteria, then the list of appointments would be sorted in order of morning, afternoon, and late afternoon to be sent out for confirmation requests, thereby minimizing the likelihood of receiving a rejected appointment request and minimizing the amount of time for treatment for each patient.

The present invention therefore provides a computer-implemented method for scheduling a referral appointment, wherein the computer-implemented method may include one or more processors configured for receiving text data corresponding to a referral request; determining patient data based on the text data comprising one or more of patient demographic data, patient complaint data, patient medication data, and patient history data; determining doctor data based on the text data comprising one or more of sending doctor data and receiving doctor data; determining a patient treatment time based on the patient data and the receiving doctor data; sending an appointment request including the patient treatment time to a computing device associated with the receiving doctor data; and receiving an appointment confirmation including the patient treatment time, wherein the appointment confirmation indicates acceptance of the appointment request.

In an embodiment, the computer-implemented method may further include one or more processors configured for identifying text fields having respective text field entries represented in a medical referral document image comprising the referral request; and generating the text data using the text fields and the respective text field entries.

In an embodiment, the patient data may be determined using a NLP engine to extract the identified text fields corresponding to a patient and identify the respective text field entries corresponding to the patient as one of either the patient demographic data, the patient complaint data, the patient medication data, or the patient history data as the patient data.

In another embodiment, the doctor data may be determined using a NLP engine to extract the identified text fields corresponding to a sending doctor and a receiving doctor and identify the respective text field entries corresponding to the sending doctor and the receiving doctor as the doctor data using the NLP engine.

In an embodiment, the computer-implemented method may further include one or more processors configured for determining doctor schedule data based on the receiving doctor data, wherein the patient treatment time is further determined based on the doctor schedule data. For example, receiving doctor data may include doctor demographic data used to identify a doctor profile associated with the receiving doctor. Once the receiving doctor profile is identified, the one or more processors may be configured to retrieve or access the doctor schedule data as made available to the network by the receiving doctor. The doctor schedule data may include dates and times of availability to accept appointments for incoming patients.

The computer-implemented method may further include one or more processors configured for determining patient schedule data based on the patient demographic data, wherein the patient treatment plan is further determined based on the patient schedule data. For example, patient demographic data may be used to identify a patient profile associated with the patient. Once the patient profile is identified, the one or more processors may be configured to retrieve or access the patient schedule data as made available to the network by the patient. The patient schedule data may include dates and times of availability to attend appointments for upcoming treatments or visits.

In an embodiment, the patient treatment time may be determined by training a machine learning model with data sets including the patient history data and receiving doctor history data; providing the patient data and the receiving doctor data to the trained machine learning model; and predicting the patient treatment time as a quantity of time represented by output data of the machine learning model.

For example, data sets may include the following values: current problem: back pain; history of problems: diabetes, Scoliosis, Herniated Disc; age: 80; previousTimeMoreThanExpected: [10,20,10]; doctorAge: 32; doctorPreviousTimeMoreThanExpected: [10,10,10]; times [morning:15,evening:20]. In this example, the input data for the data set includes patient data (e.g., current problem, history of problems, age, and previousTimeMoreThanExpected) and doctor data (e.g., doctorAge, and doctorPreviousTimesMoreThanExpected) and the output data for the data set includes data corresponding to the appointment request (e.g., times [morning:15,evening:20]). The patient time over appointment entry (“previousTimeMoreThanExpected”) may include one or more time values corresponding to a quantity of time beyond the scheduled appointment time. For example, if previous appointments were scheduled for 30 minutes, 45 minutes and 60 minutes, and the appointment durations were actually 40 minutes, 65 minutes, and 70 minutes, then the time over appointment entry may be 10 minutes, 20 minutes, and 10 minutes, respectively. Similarly, the receiving doctor previous times over appointment (“doctorPreviousTimesMoreThanExpected”) entry may include one or more time values corresponding to a quantity of time beyond the receiving doctor's treatment time. Furthermore, the output data may include time entries corresponding to the number of appointments scheduled during the morning (e.g., 15) and in the evening (e.g., 20). The output data may include various types of data, including proposed appointment times or just one optimal time. Once the receiving doctor accepts a time for the appointment, the receiving doctor's acceptance may be sent as a confirmation to the network and distributed to the appropriate parties. Output data may also include an appointment time that has been agreed upon by the receiving doctor and the patient via their respective computing devices.

The present invention will now be described in detail with reference to the Figures.

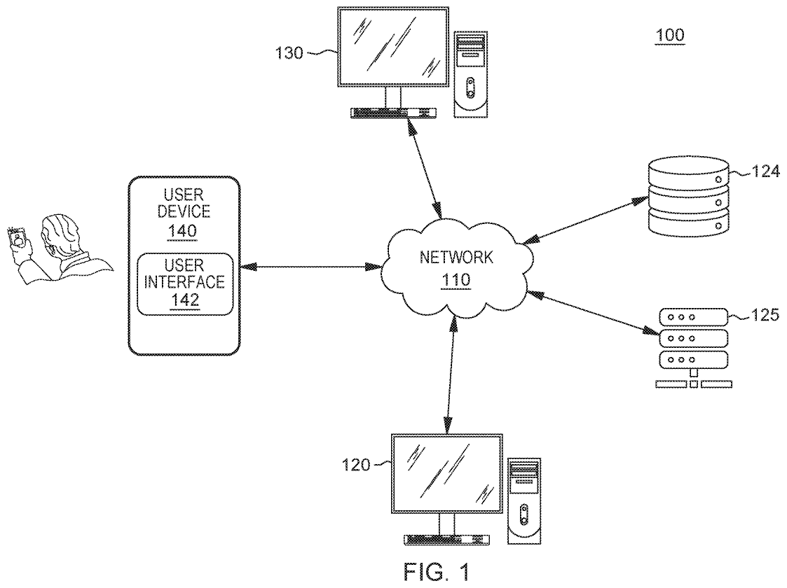

FIG. 1 depicts a block diagram of a distributed data processing environment for efficiently scheduling referral appointments, in accordance with an embodiment of the present invention. FIG. 1 provides only an illustration of one embodiment of the present invention and does not imply any limitations with regard to the environments in which different embodiments may be implemented. In the depicted embodiment, distributed data processing environment 100 includes sending doctor computing device 120, receiving doctor computing device 130, user device 140, server 125, and database 124, interconnected over network 110. Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between sending doctor computing device 120, receiving doctor computing device 130, user device 140, server 125, and database 124. Distributed data processing environment 100 may also include additional servers, computers, or other devices not shown.

Sending doctor computing device 120, receiving doctor computing device 130, and user device 140 operate to execute at least a part of a computer program for performing a diagnostic assessment. In an embodiment, sending doctor computing device 120, receiving doctor computing device 130, and/or user device 140 may be configured to send and/or receive data from one or more of the other computing device(s) via network 110. User device 140 may include user interface 142 configured to facilitate interaction between a user and user device 140. For example, user interface 142 may include a display as a mechanism to display data to a user and may be, for example, a touch screen, light emitting diode (LED) screen, or a liquid crystal display (LCD) screen. User interface 142 may also include a keypad or text entry device configured to receive alphanumeric entries from a user. User interface 142 may also include other peripheral components to further facilitate user interaction or data entry by user associated with user device 140.

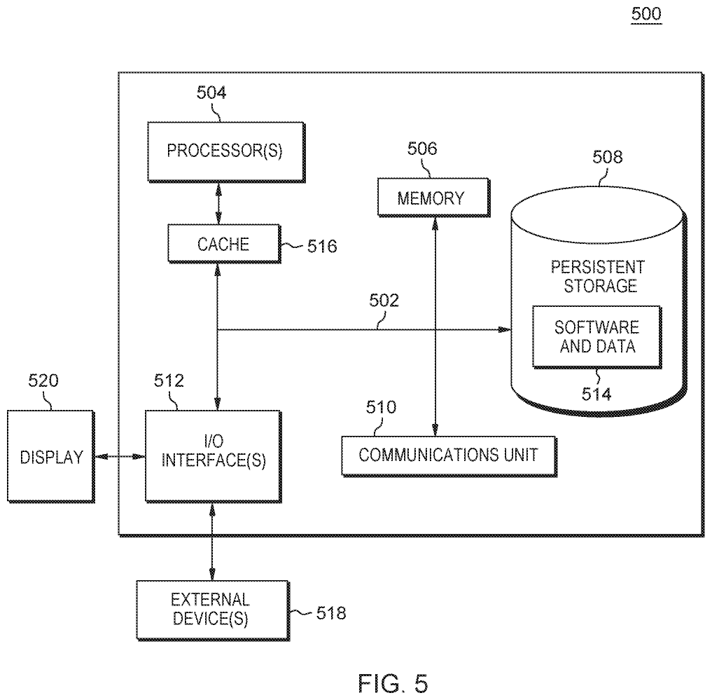

In some embodiments, sending doctor computing device 120, receiving doctor computing device 130, and/or user device 140 may be a management server, a web server, or any other electronic device or computing system capable of receiving and sending data. In some embodiments, sending doctor computing device 120, receiving doctor computing device 130, and/or user device 140 may be a laptop computer, tablet computer, netbook computer, personal computer (PC), a desktop computer, a smart phone, or any programmable electronic device capable of communicating with database 124, server 125 via network 110. Sending doctor computing device 120, receiving doctor computing device 130, and/or user device 140 may include components as described in further detail in FIG. 5.

Sending doctor computing device 120 and receiving doctor computing device 130 may be configured to receive, store, and/or process images captured on an image sensor in communication with other computing device(s) connected to network 110. For example, receiving doctor computing device 130 may be communicatively coupled to an image capture device and receive, via a communications link, data corresponding to image data captured by image capture device sensor. Sending doctor computing device 120 and receiving doctor computing device 130 may be configured to store the image data in memory or transmit the image data to database 124 and/or server 125 via network 110 for further storage and/or processing.

Database 124 operates as a repository for data flowing to and from network 110. Examples of data include patient data, doctor data, and other data that may be determined based on the patient data and the doctor data. A database is an organized collection of data. Database 124 can be implemented with any type of storage device capable of storing data and configuration files that can be accessed and utilized by sending doctor computing device 120, receiving doctor computing device 130, and/or user device 140, such as a database server, a hard disk drive, or a flash memory. In an embodiment, database 124 is accessed by sending doctor computing device 120, receiving doctor computing device 130, and/or user device 140 to store data corresponding to scheduling a referral appointment. In another embodiment, database 124 may reside elsewhere within distributed network environment 100 provided database 124 has access to network 110.

Server 125 can be a standalone computing device, a management server, a web server, or any other electronic device or computing system capable of receiving, sending, and processing data and capable of communicating with sending doctor computing device 120, receiving doctor computing device 130, user device 140, and/or database 124 via network 110. In other embodiments, server 125 represents a server computing system utilizing multiple computers as a server system, such as a cloud computing environment. In yet other embodiments, server 125 represents a computing system utilizing clustered computers and components (e.g., database server computers, application server computers, etc.) that act as a single pool of seamless resources when accessed within distributed data processing environment 100. Server 125 may include components as described in further detail in FIG. 5.

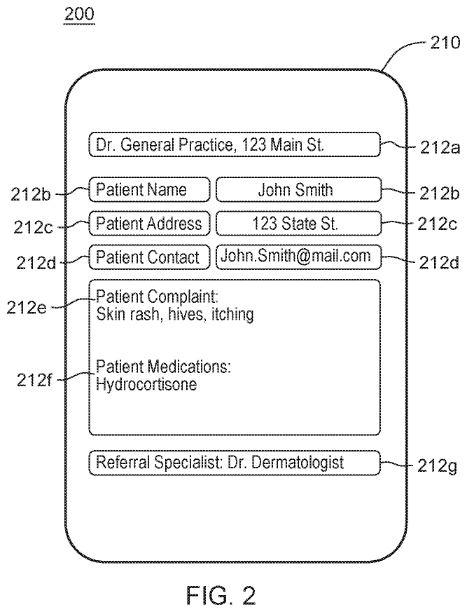

FIG. 2 depicts an example embodiment of a medical referral document image 200 to efficiently schedule referral appointments, in accordance with an embodiment of the present invention. In at least some embodiments, the image 200 may be representative of a medical referral document 210 comprising text fields and respective text field entries 212 corresponding to a referral request. The text fields and respective text field entries 212 may be scanned and captured by an image capture device and converted to text data corresponding to the text fields and the text field entries. For example, the medical referral document 210 may be scanned by an image capture device and converted to image data. One or more processors may be configured to process the image data to produce text data. Further, one or more processors may be configured for identifying the text fields and respective text fields represented in the medical referral document image 200. In other words, the one or more processors are configured to generate text data using the text fields and respective text field entries.

The one or more processors may be configured to identify text fields and respective text field entries in the medical referral document 210. Text fields and respective text field entries may be identified as patient data or doctor data. For example, patient data may include one of either patient demographic data, patient complaint data, patient medication data, or patient history data. For example, patient demographic data may include text fields and respective text field entries corresponding to “Patient Name: John Smith” 212b, “Patient Address: 123 State St.” 212c, or “Patient Contact: John.Smith@mail.com” 212d. Further, patient complaint data may include a text field and a respective text field entry corresponding to “Patient Complaint: Skin rash, hives, itching” 212e, for example. Further, patient medication data may include text fields and respective text field entries corresponding to “Patient Medications: Hydrocortisone” 212f, for example. Doctor data may be identified as sending doctor data (e.g., Dr. General Practice, 123 Main St.) 212a or receiving doctor data (e.g., Referral Specialist: Dr. Dermatologist) 212g.

In another embodiment, the medical referral document 210 may be in the form of electronic data obtained from a medical professional entering data in the text fields and the respective text field entries into a form. For example, if a medical professional prepares the medical referral document 210 on a computing device by entering data into a form, then the medical referral document 210 would be an electronic record to be sent to a receiving doctor as the referral request. Nonetheless, the one or more processors may be configured to identify text fields having respective text field entries represented in the medical referral document comprising the referral request and generating text data using the text fields and respective text field entries, as described above.

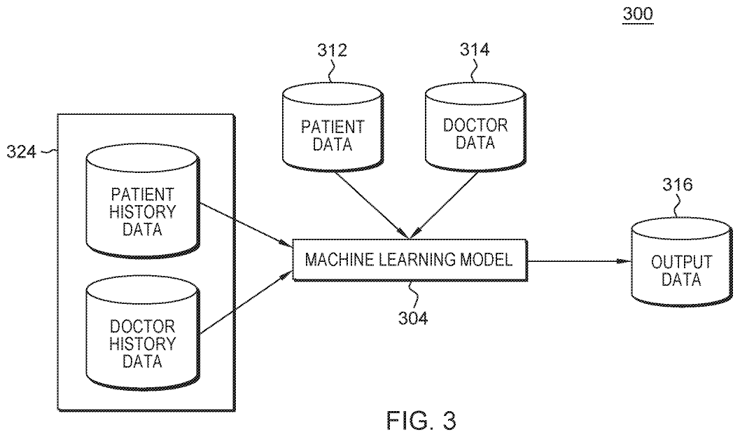

Now referring to FIG. 3, depicted is a block diagram of a system 300 for efficiently scheduling referral appointments using a machine learning model. In the depicted embodiment, the system 300 may include a machine learning model 304 configured to receive patient history data and doctor history data from database 324 to train machine learning model 304 and produce output data 316. Database 324 may include repositories for receiving and storing patient history data and doctor history data to be sent upon request to machine learning model 304. Machine learning model 304 may also be configured to receive patient data 312 and receiving doctor data 314 to produce output data 316 corresponding to a proposed appointment time for a patient to seek treatment from the receiving doctor.

Various machine learning techniques may be used to train and operate trained components to perform various processes described herein. Models may be trained and operated according to various machine learning techniques. Such techniques may include, for example, neural networks (such as deep neural networks and/or recurrent neural networks), inference engines, trained classifiers, etc. Examples of trained classifiers include Support Vector Machines (SVMs), neural networks, decision trees, AdaBoost (short for “Adaptive Boosting”) combined with decision trees, and random forests. Focusing on SVM as an example, SVM is a supervised learning model with associated learning algorithms that analyze data and recognize patterns in the data, and which are commonly used for classification and regression analysis. Given a set of training examples, each marked as belonging to one of two categories, an SVM training algorithm builds a model that assigns new examples into one category or the other, making it a non-probabilistic binary linear classifier. More complex SVM models may be built with the training set identifying more than two categories, with the SVM determining which category is most similar to input data. An SVM model may be mapped so that the examples of the separate categories are divided by clear gaps. New examples are then mapped into that same space and predicted to belong to a category based on which side of the gaps they fall on. Classifiers may issue a “score” indicating which category the data most closely matches. The score may provide an indication of how closely the data matches the category.

In order to apply the machine learning techniques, the machine learning processes themselves need to be trained. Training a machine learning component requires establishing a “ground truth” for the training examples. In machine learning, the term “ground truth” refers to the accuracy of a training set's classification for supervised learning techniques. Various techniques may be used to train the models including backpropagation, statistical learning, supervised learning, semi-supervised learning, stochastic learning, or other known techniques.

The machine learning model 304 may be configured to receive multiple input data sets and produce model output data (e.g., output data 316) as feature vectors corresponding to each data set and transmit the model output data to a series of fully connected layers. The output data 316 from the fully connected layers may be representative of a predicted or proposed appointment time. In other words, if the input data includes patient data for a patient and doctor data for a sending doctor and a receiving doctor, model output data may include a patient treatment time and date for the patient. This model output data may be in the form of a confidence score or probability for each classification representing the likelihood of a receiving doctor accepting/confirming the patient treatment time and date.

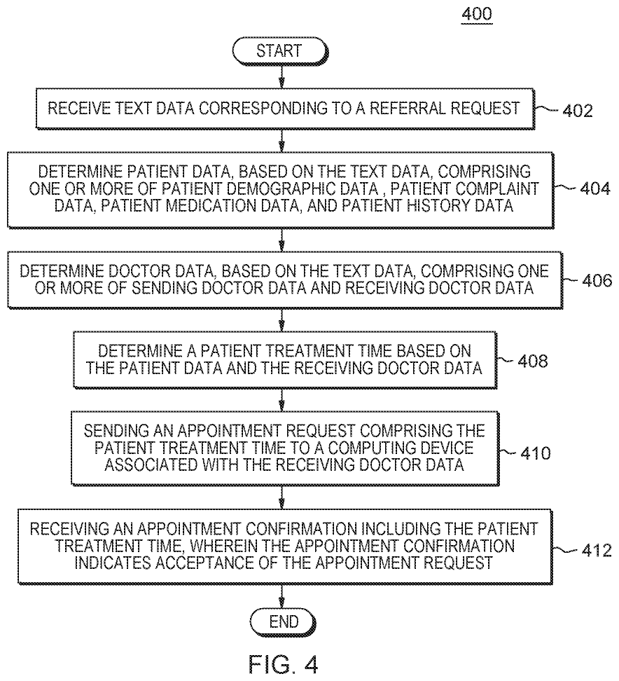

FIG. 4 depicts a flow chart of a computer-implemented method 400 for scheduling a referral appointment, in accordance with an embodiment of the present invention.

In an embodiment, computer-implemented method 400 may include one or more processors configured to receive 402 text data corresponding to a referral request. In an embodiment, the received referral request may include a medical referral document or an electronic record as described above herein. For example, text data corresponding to a referral request may be sent from a computing device associated with a sending doctor to a computing device associated with a receiving doctor via a network. A user device may also be configured to send the text data to be received by the computing device associated with a receiving doctor via network.

Further, method 400 may include one or more processors configured to determine 404 patient data based on the text data comprising one or more of patient demographic data, patient complaint data, patient medication data, and patient history data. For example, once the text data is received at computing device 120 or computing device 130, one or more processors may be configured to identify data corresponding to the patient and classify that data as patient data. Text fields and respective text field entries of a medical referral document may be identified and processed by a machine learning model to classify data corresponding to a patient as one of either patient demographic data, patient complaint data, patient medication data, or patient history data. For example, as shown in FIG. 2, patient demographic data may include text fields and respective text field entries corresponding to “Patient Name: John Smith” 212b, “Patient Address: 123 State St.” 212c, or “Patient Contact: John.Smith@mail.com” 212d. Further, patient complaint data may include a text field and a respective text field entry 212 corresponding to “Patient Complaint: Skin rash, hives, itching” 212e, for example. Further, patient medication data may include text fields and a respective text field entries 212 corresponding to “Patient Medications: Hydrocortisone” 212f, for example.

Further, method 400 may further include one or more processors configured to determine 406 doctor data comprising one or more of sending doctor data and receiving doctor data based on the text data. For example, if a text field and respective text field entry of a medical referral document identifies words or phrases corresponding to a doctor, medical professional, hospital, or other related medical proper nouns, then the one or more processors may be configured to identify the text field and respective text field entry as doctor data.

Sending doctor data may be classified as doctor data corresponding to the medial professional or medical facility identified as the originator of the referral request or the medical referral document. For example, sending doctor data may be identified as a text field and respective text field entry having location or content characteristics within the medical referral document. For instance, a text field and respective text field entry appearing at a first location (e.g., “Dr. General Practice, 123 Main St.” top of the page) 212a may correspond to the sending doctor or at a second location (e.g., “Referral Specialist: Dr. Dermatologist” bottom of the page) 212g may correspond to the receiving doctor. Further, a text field and respective text field entry having text including sending doctor characteristics (e.g., general practice, primary care physician) may correspond to the sending doctor (e.g., “Dr. General Practice, 123 Main St.”) and text including receiving doctor characteristics (e.g., referral, specialist) may correspond to the receiving doctor (e.g., “Referral Specialist: Dr. Dermatologist”). Other rules and guidelines may be provided to configure the one or more processors to identify and classify sending doctor data, receiving doctor data, and other classifiable data types within the medical referral document. Receiving doctor data may be classified as doctor data corresponding to the medical professional or medical facility identified as the intended recipient of the medical referral document.

Further, method 400 may include one or more processors configured to determine 408 patient treatment time based on the patient data and the receiving doctor data, as described above herein. The patient treatment time may be determined by training a machine learning model with data sets including the patient history data and receiving doctor history data; providing the patient data and the receiving doctor data to the trained machine learning model; and predicting the patient treatment time as a quantity of time represented by output data of the machine learning model. The output data may include various types of data, including proposed appointment times or just one optimal time. Once the receiving doctor accepts a time for the appointment, the receiving doctor's acceptance may be sent as a confirmation to the network and distributed to the appropriate parties. Output data may also include an appointment time that has been agreed upon by the receiving doctor and the patient via their respective computing devices.

Further, method 400 may include one or more processors configured to send 410 an appointment request including the patient treatment time to a computing device associated with the receiving doctor data, as described above herein. The one or more processors may be configured to transmit data corresponding to an appointment request to a computing device associated with the receiving doctor. The appointment request may be generated by processing text data corresponding to a referral request. The appointment request may also come in the form of a telephone call from a user device associated with the patient to a computing device associated with the receiving doctor. The text data may include patient data and doctor data, wherein the doctor data may include sending doctor data and receiving doctor data.

Further, method 400 may include one or more processors configured to receive 412 an appointment confirmation including the patient treatment time, wherein the appointment confirmation indicates acceptance of the appointment request, as described above herein. For example, if a computing device associated with a receiving doctor receives an input corresponding to the receiving doctor accepting the appointment request, then the computing device may be configured to transmit the input as an appointment confirmation to the network for communication to the user device associated with the patient profile and the patient. Thus, the one or more processors may receive 412 the appointment confirmation transmitted by the computing device associated with the receiving doctor.

FIG. 5 depicts a block diagram of a computing device of distributed computing environment, in accordance with an embodiment of the present invention. FIG. 5 depicts a block diagram of computing device 500 suitable for server 125, computing device 120, computing device 130, and user device 140 in accordance with an illustrative embodiment of the present invention. It should be appreciated that FIG. 5 provides only an illustration of one implementation and does not imply any limitations with regard to the environments in which different embodiments may be implemented. Many modifications to the depicted environment may be made.

Computing device 500 includes communications fabric 502, which provides communications between cache 516, memory 506, persistent storage 508, communications unit 510, and input/output (I/O) interface(s) 512. Communications fabric 502 can be implemented with any architecture designed for passing data and/or control information between processors (such as microprocessors, communications and network processors, etc.), system memory, peripheral devices, and any other hardware components within a system. For example, communications fabric 502 can be implemented with one or more buses or a crossbar switch.

Memory 506 and persistent storage 508 are computer readable storage media. In this embodiment, memory 506 includes random access memory (RAM). In general, memory 506 can include any suitable volatile or non-volatile computer readable storage media. Cache 516 is a fast memory that enhances the performance of computer processor(s) 504 by holding recently accessed data, and data near accessed data, from memory 506.

Programs may be stored in persistent storage 508 and in memory 506 for execution and/or access by one or more of the respective computer processors 504 via cache 516. In an embodiment, persistent storage 508 includes a magnetic hard disk drive. Alternatively, or in addition to a magnetic hard disk drive, persistent storage 508 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 508 may also be removable. For example, a removable hard drive may be used for persistent storage 508. Other examples include optical and magnetic disks, thumb drives, and smart cards that are inserted into a drive for transfer onto another computer readable storage medium that is also part of persistent storage 508.

Communications unit 510, in these examples, provides for communications with other data processing systems or devices. In these examples, communications unit 510 includes one or more network interface cards. Communications unit 510 may provide communications through the use of either or both physical and wireless communications links. Programs, as described herein, may be downloaded to persistent storage 508 through communications unit 510.

I/O interface(s) 512 allows for input and output of data with other devices that may be connected to sending computing device 120, receiving computing device, or user device 140. For example, I/O interface 512 may provide a connection to external devices 518 such as image sensor, a keyboard, a keypad, a touch screen, and/or some other suitable input device. External devices 518 can also include portable computer readable storage media such as, for example, thumb drives, portable optical or magnetic disks, and memory cards. Software and data 514 used to practice embodiments of the present invention can be stored on such portable computer readable storage media and can be loaded onto persistent storage 508 via I/O interface(s) 512. I/O interface(s) 512 also connect to a display 520.

Display 520 provides a mechanism to display data to a user and may be, for example, a computer monitor.

Software and data 514 described herein is identified based upon the application for which it is implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for scheduling a referral appointment, comprising:
receiving, by one or more processors, text data corresponding to a referral request;
determining, by the one or more processors, patient data, based on the text data, comprising one or more of patient demographic data, patient complaint data, patient medication data, and patient history data;
determining, by the one or more processors, doctor data, based on the text data, comprising receiving doctor data;
determining, by the one or more processors, a patient treatment time based on the patient data and the receiving doctor data;
sending, by the one or more processors, an appointment request including the patient treatment time to a computing device associated with the receiving doctor data; and
receiving, by the one or more processors, an appointment confirmation including the patient treatment time, wherein the appointment confirmation indicates acceptance of the appointment request.

2. The computer-implemented method of claim 1, further comprising:
identifying, by the one or more processors, text fields having respective text field entries represented in a medical referral document image comprising the referral request; and
generating, by the one or more processors, the text data using the text fields and the respective text field entries.

3. The computer-implemented method of claim 2, wherein determining the patient data comprises:
using, by the one or more processors, a Natural Language Processor (NLP) engine to: extract the identified text fields corresponding to a patient and identify the respective text field entries corresponding to the patient as one of the patient demographic data, the patient complaint data, the patient medication data, or the patient history data as the patient data.

4. The computer-implemented method of claim 2, wherein determining the doctor data comprises:
extracting, by the one or more processors, the identified text fields corresponding to a sending doctor and a receiving doctor using a Natural Language Processor (NLP) engine; and
identifying, by the one or more processors, the respective text field entries corresponding to the sending doctor and the receiving doctor as the doctor data using the NLP engine.

5. The computer-implemented method of claim 1, further comprising:
determining, by the one or more processors, doctor schedule data based on the receiving doctor data, wherein the patient treatment time is further determined based on the doctor schedule data.

6. The computer-implemented method of claim 1, further comprising:
determining, by the one or more processors, patient schedule data based on the patient demographic data, wherein the patient treatment plan is further determined based on the patient schedule data.

7. The computer-implemented method of claim 1, wherein determining the patient treatment time comprises:
training, by the one or more processors, a machine learning model with data sets including the patient history data and receiving doctor history data;
providing, by the one or more processors, the patient data and the receiving doctor data to the trained machine learning model; and
predicting, by the one or more processors, the patient treatment time as a quantity of time represented by output data of the machine learning model.

8. A computer program product for scheduling a referral appointment, the computer program product comprising:
one or more computer readable storage media and program instructions stored on the one or more computer readable storage media, the program instructions comprising:
program instructions to receive text data corresponding to a referral request;
program instructions to determine patient data based on the text data, the patient data comprising one or more of patient demographic data, patient complaint data, patient medication data, and patient history data;
program instructions determine doctor data based on the text data, the doctor data comprising receiving doctor data;
program instructions to determine a patient treatment time based on the patient data and the receiving doctor data;
program instructions to send an appointment request including the patient treatment time to a computing device associated with the receiving doctor data; and
program instructions to receive an appointment confirmation including the patient treatment time, wherein the appointment confirmation indicates acceptance of the appointment request.

9. The computer program product of claim 8, further comprising:
program instructions to identify text fields having respective text field entries represented in a medical referral document image comprising the referral request; and
program instructions to generate the text data using the text fields and the respective text field entries.

10. The computer program product of claim 9, wherein determining the patient data comprises:
program instructions to use a Natural Language Processing (NLP) engine to:
extract the identified text fields corresponding to a patient; and
identify the respective text field entries corresponding to the patient as one of either the patient demographic data, the patient complaint data, the patient medication data, or the patient history data as the patient data.

11. The computer program product of claim 9, wherein determining the doctor data comprises:
program instructions to use a Natural Language Processor (NLP) engine to:
extract the identified text fields corresponding to a sending doctor and a receiving doctor; and
identify the respective text field entries corresponding to the sending doctor and the receiving doctor as the doctor data.

12. The computer program product of claim 8, further comprising:
program instructions to determine doctor schedule data based on the receiving doctor data, wherein the patient treatment time is further determined based on the doctor schedule data.

13. The computer program product of claim 8, further comprising:
program instructions to determine patient schedule data based on the patient demographic data, wherein the patient treatment plan is further determined based on the patient schedule data.

14. The computer program product of claim 8, wherein determining the patient treatment time comprises:
program instructions to train a machine learning model with data sets including the patient history data and receiving doctor history data;
program instructions to provide the patient data and the receiving doctor data to the trained machine learning model; and
program instructions to predict the patient treatment time as a quantity of time represented by output data of the machine learning model.

15. A computer system for scheduling a referral appointment, the computer system comprising:
one or more computer processors;
one or more computer readable storage media;
program instructions stored on the one or more computer readable storage media for execution by at least one of the one or more processors, the program instructions comprising:
program instructions to receive text data corresponding to a referral request;
program instructions to determine patient data based on the text data, the patient data comprising one or more of patient demographic data, patient complaint data, patient medication data, and patient history data;
program instructions determine doctor data based on the text data, the doctor data comprising receiving doctor data;
program instructions to determine a patient treatment time based on the patient data and the receiving doctor data;
program instructions to send an appointment request including the patient treatment time to a computing device associated with the receiving doctor data; and
program instructions to receive an appointment confirmation including the patient treatment time, wherein the appointment confirmation indicates acceptance of the appointment request.

16. The computer system of claim 15, further comprising:
program instructions to identify text fields having respective text field entries represented in a medical referral document image comprising the referral request; and
program instructions to generate the text data using the text fields and the respective text field entries.

17. The computer system of claim 16, wherein determining the patient data comprises:
program instructions to use a Natural Language Processing (NLP) engine to:
extract the identified text fields corresponding to a patient; and
identify the respective text field entries corresponding to the patient as one of either the patient demographic data, the patient complaint data, the patient medication data, or the patient history data as the patient data.

18. The computer system of claim 16, wherein determining the doctor data comprises:
program instructions to use a Natural Language Processor (NLP) engine to:
extract the identified text fields corresponding to a sending doctor and a receiving doctor; and
identify the respective text field entries corresponding to the sending doctor and the receiving doctor as the doctor data.

19. The computer system of claim 15, further comprising:
program instructions to determine doctor schedule data based on the receiving doctor data, wherein the patient treatment time is further determined based on the doctor schedule data; and
program instructions to determine patient schedule data based on the patient demographic data, wherein the patient treatment plan is further determined based on the patient schedule data.

20. The computer system of claim 15, wherein determining the patient treatment time comprises:
program instructions to train a machine learning model with data sets including the patient history data and receiving doctor history data;
program instructions to provide the patient data and the receiving doctor data to the trained machine learning model; and
program instructions to predict the patient treatment time as a quantity of time represented by output data of the machine learning model.
